(19) United States
(12) Patent Application Publication
(10) Pub. No.: US 2019/0321209 A1
(43) Pub. Date: Oct. 24, 2019

(54) PEDITRACK

(71) Applicant: Angela A. Hatzis, Floral Park, NY (US)

(72) Inventor: Angela A. Hatzis, Floral Park, NY (US)

(21) Appl. No.: 15/958,240

(22) Filed: Apr. 20, 2018

(51) Int. Cl. A61F 5/01; A43B 7/26; A61F 5/14

(52) U.S. Cl. CPC A61F 5/019; A43B 7/26; A43B 7/1415; A61F 5/0127; A61F 5/14

(57) ABSTRACT

The invention provides bunion corrector footwear. The bunion corrector footwear comprises a bunion corrector that includes a strip of rigid material to support at least two toes. A cushiony gel separator is placed between at least two toes. Further, a metatarsal support attached on the foot wear to support the bunion corrector. The footwear herein will provide support for bunion prevention and also the healing of existing bunions without any surgical intervention.

### TECHNICAL FIELD

The present disclosure relates to bunion corrector footwear. More specifically, the invention relates to footwear that provides support for bunion prevention and also the healing of existing bunions without any surgical intervention.

### BACKGROUND OF THE DISCLOSURE

Generally, people use footwears such as athletic shoes or sports shoes, sandals, flip-flops, slippers etc., often while walking, running, or undertaking physical work outs. The footwear protects the foot and it is a direct walking means essentially for humans.

A bunion, or hallux valgus, is the tilting of the toe away from the mid-line of the foot. While bunions may occur in any toe, they typically occur in the big toe and only very occasionally in the little toe. Bunions at the base of the big toe usually begin when the big toe starts moving toward the smaller toes, such as when tight, pointed shoes are worn. This crowding puts pressure on the joint, pushing it outward. The portions of the bones intersecting at and comprising the joint therefore become hyper-rotated, resulting in the joint moving outwardly as a result of the stresses upon it. The movement of the joint in this outward direction initiates the formation of a bunion as the joined bones are no longer collinear.

As noted above, bunions arise from mechanical instability within the foot, resulting in a misalignment of the bones therein and are typically characterized by the big toe drifting either over or under the second toe. As a bunion deformity progresses, a characteristic bump forms and grows behind the inside of the big toe. The act of walking requires the big toe to support a great deal of the body's weight and therefore actuates the progression of the bunion. As the bunion increases in severity, the condition may be complicated by the generation of other associated foot problems, such as hammertoes, painful calluses on the bottom of the foot, arch pain and the like.

Pain from a bunion can range from mild to severe, making it difficult to walk in normal shoes, especially high-heeled shoes. The skin and deeper tissues around the bunion may also become swollen or inflamed. Moreover, the other toes may be affected by the bunion, such as by result of pressure from the big toe urging the lesser toes inwardly. Further, toenails may begin to grow into the sides of the nail bed. Likewise, the smaller toes can develop corns and/or become bent (hammertoes) and/or calluses may form on the bottom of the foot. Bunion treatments vary depending on the severity of pain and deformity related to the bunion. When left untreated, bunions tend to grow larger and, usually, more painful. One common bunion treatment is to pad the bunion. While effective in diminishing the pain associated with the bunion, padding does not address the cause of the bunion. Likewise, switching from poorly fitting and constrictive shoes may lessen the pain from the bunion, but do not reverse the bunion condition. Likewise, medications, such as anti-inflammatory drugs or cortisone injections may be administered to ease pain and inflammation caused by joint deformities. However, such medications do not correct or reverse such ongoing joint deformities.

### BRIEF SUMMARY

The present invention overcomes the drawbacks in the prior art and provides a bunion corrector footwear. The footwear comprises a sole base, a bunion corrector attached to the sole base, wherein the bunion corrector includes a strip of rigid material to support at least two toes. The footwear further comprises a cushiony gel separator placeable between at least two toes and a metatarsal support attached on the foot wear to support the bunion corrector.

In some example embodiment, the footwear comprises a thong.

In some example embodiment, the cushiony gel separator is configured to provide separation between the at least two toes to correct bunions.

In some example embodiment, the size and shape of the metatarsal support depends upon the size and shape of the bunion corrector, respectively.

In some example embodiment, the footwear further comprises an anatomical arch support.

In some example embodiment, the footwear further comprises a heel cup for support; the footwear enables the toes and the feet to move naturally while keeping the alignment.

In some embodiment, the cushiony gel separator is an integral part of the footwear.

In some example embodiment, the footwear further comprises flexible engineered padded foot beds to alleviate strain on the plantar fascia, and increase blood circulation.

In some example embodiment, the footwear further comprises a toe box to accommodate built in gel toe spacers.

In some example embodiments, the bunion corrector is independently adjustable to snugly engage the at least two toes.

### BRIEF DESCRIPTION OF THE DRAWINGS

Having thus described example embodiments of the invention in general terms, reference will now be made to the accompanying drawings, which are not necessarily drawn to scale, and wherein:

FIG. 1 illustrates the footwear with a metatarsal support, in accordance with an example embodiment;

FIG. 2 illustrates the footwear with a bunion corrector, in accordance with an example embodiment; and

FIG. 3 and FIG. 4 illustrate the footwear with a cushiony gel toe separator, in accordance with an example embodiment.

### DETAILED DESCRIPTION

Some embodiments of the present invention will now be described more fully hereinafter with reference to the accompanying drawings, in which some, but not all, embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Like reference numerals refer to like elements throughout. Also, reference in this specification to “one embodiment” or “an embodiment” means that a particular feature, structure, or characteristic described in connection with the embodiment is included in at least one embodiment of the present disclosure. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity, but rather denote the presence of at least one of the referenced item. Moreover, various features are described which may be exhibited by some embodiments and not by others.

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present disclosure. Further, it is to be understood that the phraseology and terminology employed herein are for the purpose of the description and should not be regarded as limiting. Any heading utilized within this description is for convenience only and has no legal or limiting effect.

Footwear with bunion corrector is provided herein in accordance with an example embodiment for bunion prevention and also the healing of existing bunions without any surgical intervention.

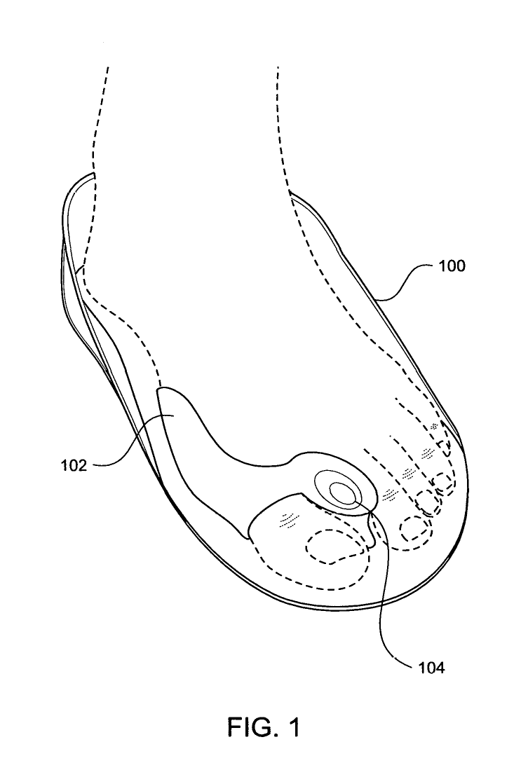

FIG. 1 illustrates a footwear 100 with a bunion corrector 102, in accordance with an example embodiment. In some example embodiments, the footwear 100 may include, but not limited to flip-flops, slippers, sandals, shoes, loafers, boots or oxfords etc. In some example embodiments, the bunion corrector 102 may include a strip of rigid material to support at least two toes.

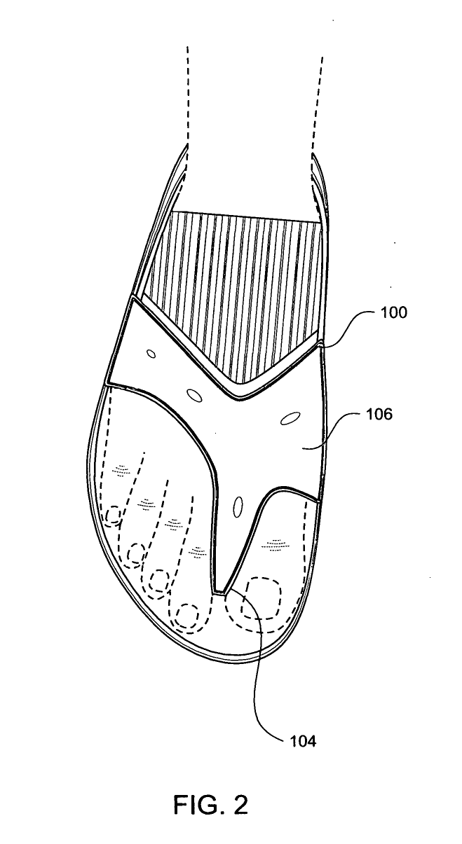

FIG. 2 illustrates the footwear 100 with a bunion corrector, in accordance with an example embodiment. In some example embodiment, the footwear 100 may include flip-flops, slippers or sandals all of which would have a built-in bunion corrector 102. In some example embodiment, an inch toe splint, which will be built into the flip-flops, consistent of a thong, the cushiony gel toe separator 104, between the big toe and the second toe. In some example embodiment, the cushiony gel toe separators 104 will comfortably keep the toes separated to correct bunions and promote proper foot alignment. By separating the toes, footwear 100 will reduce rubbing and ease discomfort caused by bunions without having to undergo debilitating surgery. In some example embodiments, a metatarsal support 106 may be attached on the footwear 100 to support the bunion corrector 102.

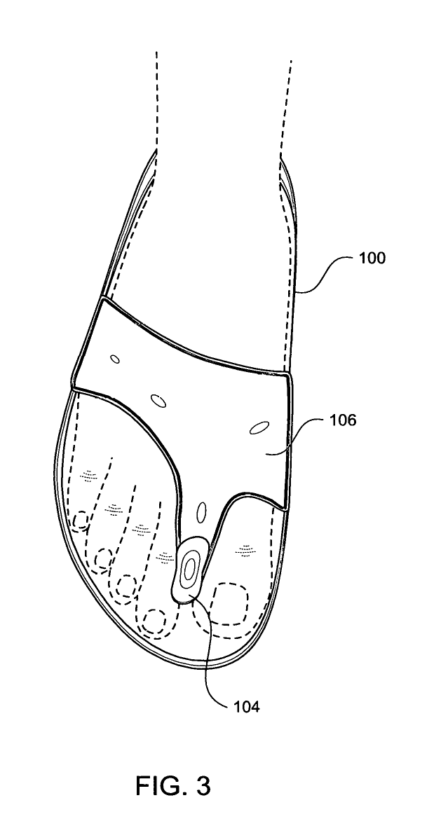

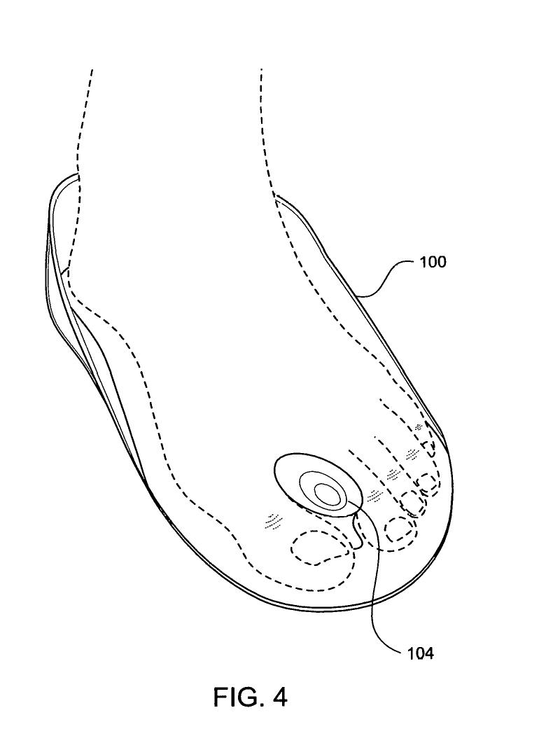

FIG. 3 and FIG. 4 illustrate the footwear with a cushiony gel toe separator, in accordance with an example embodiment. In some example embodiment, the footwear 100 may comprise a thong. In some example embodiment, the cushiony gel separator 104 keeps the at least two toes separated to correct bunions and promotes the foot alignment. In other words, the cushiony gel separator 104 provides separation between the at least two toes. The size and shape of the metatarsal support 106 may be proportional to the size and shape of the bunion corrector 102.

In some example embodiment, the footwear 100 further comprises an anatomical arch support. In some example embodiment, the footwear 100 further comprises a heel cup for support. The footwear 100 enables the toes and the feet to move naturally while keeping the alignment.

In some embodiment, the cushiony gel separator 102 forms the integral part of the footwear 100. In some example embodiment, the at least two toes include a big toe and a second toe. The footwear 100 further comprises a flexible engineered padded foot beds to alleviate strain on the plantar fascia and increase blood circulation. The footwear 100 further comprises a toe box to accommodate build in gel toe spacers. In some example embodiments, the bunion corrector 102 is independently adjustable to snugly engage the at least two toes.

In some example embodiments, the toe separators will prevent toe rubbing that will result in reducing the risk of bunions, promote healing of existing ones and prevent corns developing between the toes. For existing corns, separating the toes may allow the skin to recover. In some example embodiments, the flip-flops, slippers, sandals may have two types of widths of their supportive band on top according to individual preference. In some example embodiment, the flip-flops, slippers and sandals with a built-in bunion correction splint will be comfortably worn all day, as it will enable the toes and the feet to move naturally while keeping their alignment.

In some example embodiments, the footwear 100 will ease the discomfort, pressure and inflammation caused by bunions and will provide amazing relief all day. Further, the footwear 100 will be manufactured for women and men as it is geared to provide comfort for both men and women.

The flip-flops, slippers and sandals will be made with the finest quality of materials. One hundred percent medical materials, with high-quality rubber which will produce a perfect product. The finest grade materials will definitely reduce the discomfort of a bunion and make every day walking or exercising, in or outside the house, a fun activity with the not much effort.

In some example embodiment, the footwear 100 may be designed to use in all gym and other fitness centers. In some example embodiments, the footwear 100 with a built-in bunion relief splint secured inside the shoe so that walking dancing and exercising will be secure for the toes and comfortable. The built-in bunion splint will be build inside the sole of the leather shoes or sneakers. That will help the individual avoid discomfort, pressure and inflammation caused by bunions while providing relief and keeping the alignment of the toes at all time, in a very discreet way.

In some example embodiments, the foot bed of the footwear 100 will be made with high quality material in order to provide impact absorption, which would reduce the stress for feet and knees and lower back. It would also provide superior arch support and moisture resistance for prolonged use.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

I claim:

1. A footwear comprising:
a sole base;
a bunion corrector attached to the sole base, wherein the bunion corrector includes a strip of rigid material to support at least two toes;
a cushiony gel separator placeable between said at least two toes; and
a metatarsal support attached on the base sole to support the bunion corrector.

2. The footwear of claim 1, wherein the bunion corrector comprise a thong.

3. The footwear of claim 1, wherein the cushiony gel separator is configured to provide separation between the at least two toes to correct bunions.

4. The footwear of claim 1, wherein size and shape of the metatarsal support is proportional to the size and shape of the bunion corrector, respectively.

5. The footwear of claim 1, further comprising an anatomical arch support.

6. The footwear of claim 1, further comprising a heel cup.

7. The footwear of claim 1, wherein the cushiony gel separator is an integral part of the footwear.

8. The footwear of claim 1, further comprising flexible engineered padded foot beds to alleviate strain on the plantar fascia and increase blood circulation.

9. The footwear of claim 1, further comprising a toe box to accommodate built-in gel toe spacers.

10. The footwear of claim 1, wherein the bunion corrector is independently adjustable to snugly engage the at least two toes.
